(19) United States
(12) Patent Application Publication
(10) Pub. No.: US 2011/0087248 A1
(43) Pub. Date: Apr. 14, 2011

(54) Flexor Tendon Suture Passer

(76) Inventor: Dennis L. Steffen, Tavernier, FL (US)

(21) Appl. No.: 12/904,221

(22) Filed: Oct. 14, 2010

Related U.S. Application Data
(60) Provisional application No. 61/251,353, filed on Oct. 14, 2009.

(51) Int. Cl. A61B 17/04

(52) U.S. Cl. 606/148

(57) ABSTRACT

A suture passer system and method are provided. The system includes a dual shaft suture passer. The suture passer is especially configured and contoured to aid in the retrieval of sutures during a flexor tendon repair procedures.

### CROSS REFERENCES TO RELATED APPLICATIONS

This application claims the benefit under 35 U.S.C. §119(e) of U.S. Provisional Application No. 61/251,353, filed on Oct. 14, 2009, the complete disclosure of which is incorporated herein by reference in its entirety.

### FIELD OF THE INVENTION

The present invention relates to a suture passer and method for repair of the flexor tendon.

### INTRODUCTION

Conventional suture passers are not designed to address the issues and requirements associated with the repair of the flexor tendon. In addition to having technical expertise, experienced hand surgeons must have precise knowledge of flexor tendon anatomy to guide appropriate treatment of injuries to the flexor tendon system.

One method of securing the flexor tendon together is by the use of sutures. After the two end of the lacerated tendon have been identified. The tendon is pulled up thought an opening in the flexor tendon sheath a suture is then connected to this end. The suture retriever is used to pull the free end of the sutured tendon back through the sheathing positioning it adjacent to the other free end of the tendon.

### BRIEF DESCRIPTION

The invention provides a system and method for tendon repair, especially flexor tendon, in acute injury. The system includes a suture passer. The suture passer has a dual tapered shaft portion and handle portion. The tapered shafts have a convex upper surface and an opposed convex under surface with a loop in the distal tip. The handle on the proximal end of the shaft provides for manipulation of the shaft when it is inserted in the sheathing. The dual shafts are gently covered from handle on the proximal end to the tip on the distal end allowing for easier insertion in the sheathing and tendon channels while providing a more ergonomic shape better suited to the anatomy. The loop in the tip located in the distal region and accommodates the suture. The length and thickness of the shafts is also sized proportionately allowing for minimal trauma during the repair procedure in addition the shafts posses the appropriate amount of flexibility.

This device is proposed for the atraumatic retrieval of sutures within the flexor tendon sheath. The device is intended but not limited to specifically, to address the anatomy of the hand and fingers and the requirements of the flexor tendons within the confines of the flexor tendon sheath during surgical repair of lacerated tendons.

In the distal palm and the entire finger, the flexor tendons slide within fibro-osseous tunnel. The fibrous component of the tunnel is referred to as the flexor tendon sheath. The sheath is made of individual tunnels called pulleys. The repair of a lacerated tendon within the flexor tendon sheath is a technical challenge to any surgeon. It is frequently advantageous to suture the transected end of a tendon and then retrieve the suture first. Once the suture is retrieved, the tendon can be pulled through the sheath to the site of the repair. Most surgeons utilize less than adequate devices such as rubber or plastic catheters for tendon or suture retrieval (Sourmelis S G, McGrouther D A: Retrieval of the retracted flexor tendon. J Hand Surg 1987; 12B:109-111). Some surgeons recommend a loop made of malleable surgical wire; the wire loop is twisted many times to give it the shape to be used as a suture retriever. (B. Jagannath Kamath, Praveen Bhardwaj. A Simple, semirigid, and surgeon-friendly tendon retriever and flexor sheath dilator. J Hand Surg 2007; 32A:269-273). Yet another approach is shown in patent application no. 20100137883, Eduardo Gonzalez-Hernandez which suggests a suture retriever/dilator fabricated from a thermal plastic good only for single use.

In one embodiment, the invention provides a suture passer which has a suitably tear drop shape to the handle that is non-intrusive and allows the surgeon to manipulate the device and a tubular shaped tapered dual shaft portion with a convex upper surface and convex under surface. The shaft tapers in thickness from the tip to the handle region, thinner at the tip to thicker at the handle. The shaft also has a loop in the most distal region. The dual shafts gently curve from the very distal tip to the very proximal tip of the handle. The advantages of a gently curved shaft is to find its way as it is introduced and advanced into the flexor tendon sheath, where the sheath is empty as in the area of a lacerated and retracted tendon. In addition having a curved tip to find its way as it is introduced and advanced into the flexor tendon sheath, where the sheath surrounds a tendon; its design allows the suture retriever to fit very naturally without trauma to the tendon or the sheath surrounding the tendon as it is introduced and advanced into the flexor tendon sheath, where the flexor digitorum superficialis hiatus is intact; its design allows for easy entry into the hiatus of the flexor digitorum superficialis without trauma to this structure or the mesotenon. This is relevant when repairing the flexor digitorum profundus tendon.

The dual shaft configuration provides a naturally occurring curvature element, in its longitudinal axis to match the convex outline of the volar aspect of the proximal and middle phalanx where the tightest pulleys make suture retrieval more challenging

Other advantages and a better appreciation of the specific adaptations, variations, and physical attributes of the invention will be gained upon an examination of the following detailed description of the invention, taken in conjunction with the accompanying drawings.

### BRIEF DESCRIPTION OF THE DRAWINGS

The invention may be better understood and appreciated by reference to the detailed description of specific embodiments presented herein in conjunction with the accompanying drawings of which:

FIG. 1 is a top plan view of suture passer configuration with looped tip and looped tear drop handle;

FIG. 2 is a side plan view of suture passer configuration with handle and tip at gentle curve of shafts:

FIG. 3 depicts a cross sectional view of suture passer shaft configuration with naturally occurring convex upper surface and convex under surface;

FIG. 4 illustrates a perspective view of the suture passer configuration with the looped tip and tear drop handle;

FIG. 5 illustrates a perspective view of the suture passer configuration with a solid tear drop handle;

### DETAILED DESCRIPTION

A suture passer embodying the principles of the invention is provided. The system includes a suture passer.

Before any embodiments of the invention are explained in detail, it is to be understood that the invention is not limited in its application to the details of the structure and function set forth in the following description or illustrated in the appended drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. “Comprising” also encompasses the terms “consisting of” and “consisting essentially of,” The use of “consisting essentially of” means, e.g., that a method may include additional steps, but only if the additional steps do not materially alter the basic and novel characteristics of the claimed method. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

No admission is made that any reference, including any patent or patent document, cited in this specification constitutes prior art. In particular, it will be understood that unless otherwise stated, reference to any document herein does not constitute an admission that any of these documents forms part of the common general knowledge in the art in the United States or in any other country. Any discussion of the references states what the author asserts and the applicant reserves the right to challenge the accuracy and pertinence of any of the documents cited herein.

As used in this specification and the appended claims, the singular forms “a,” “an,” and “the” include plural referents unless the content clearly dictates otherwise. As used in this specification and the appended claims, the term or is generally employed in its sense including “and/or” unless the content clearly dictates otherwise.

Unless otherwise noted, technical terms are used according to conventional usage. However, as used herein, the following definitions may be useful in aiding the skilled practitioner in understanding the invention. Such definitions shall be applied, unless a different definition is given in the claims or elsewhere in this specification.

As used herein, the term “loop” or “aperture” is meant to refer a loop or aperture of a suture passer that is configured to accommodate sutures of varying sizes.

In view of the foregoing disadvantages inherent in conventional suture passers, the invention provides a novel system and method for repair of flexor tendons. The invention provides an instrument specifically designed and proportioned for use in this area. However the device is capable of being proportioned for use in other areas of the anatomy such as upper and lower extremities e.g. the distal bicep tendon repair.

The device 10 here proposed is unique to the anatomy of the hand but could easily be adapted for other areas of the anatomy.

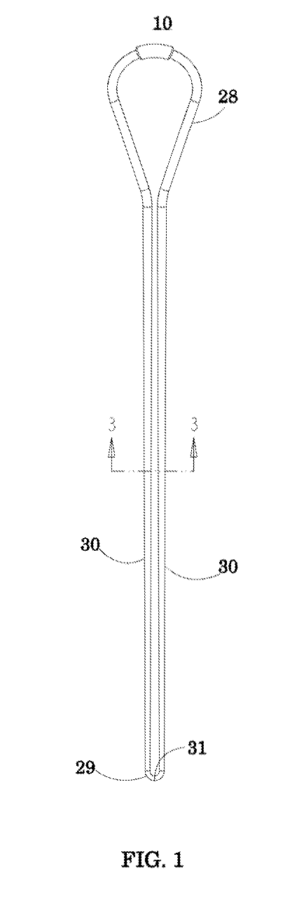

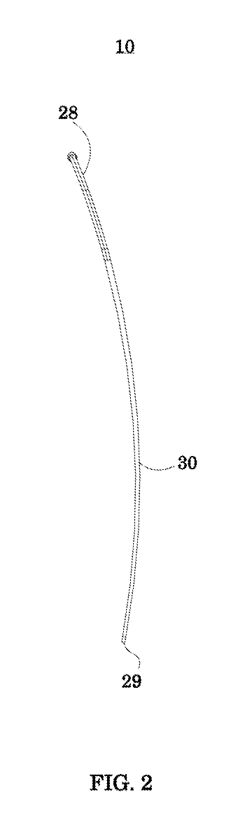

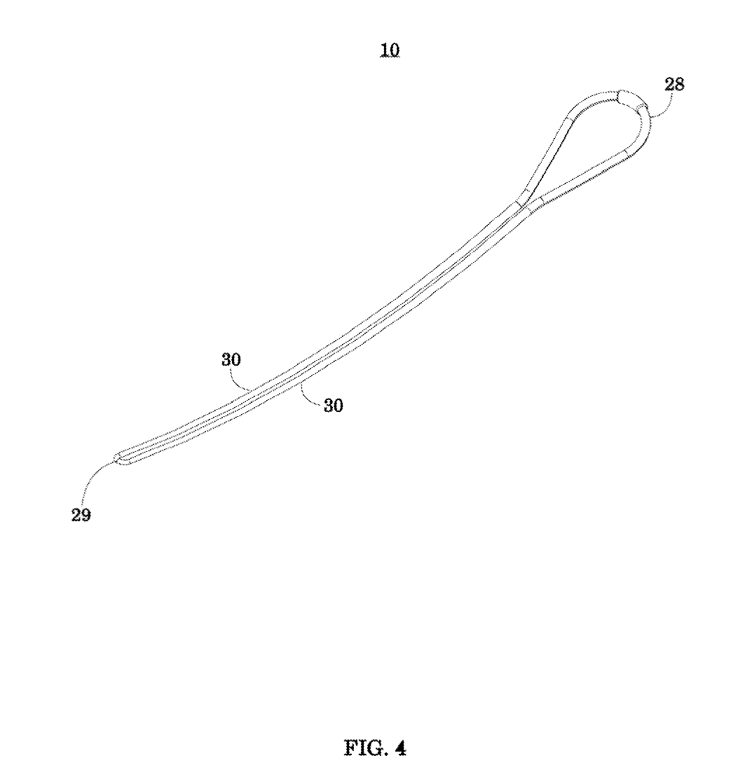

Reference is now made to FIGS. 1-5 in which a flexor tendon suture pa designated by reference numeral 10, in accordance with the invention is shown. System 10 includes a proximal handle portion 28 for manipulation of the instrument with a looped distal tip 29 centered on the minor diameter of a tapered dual shaft 30.

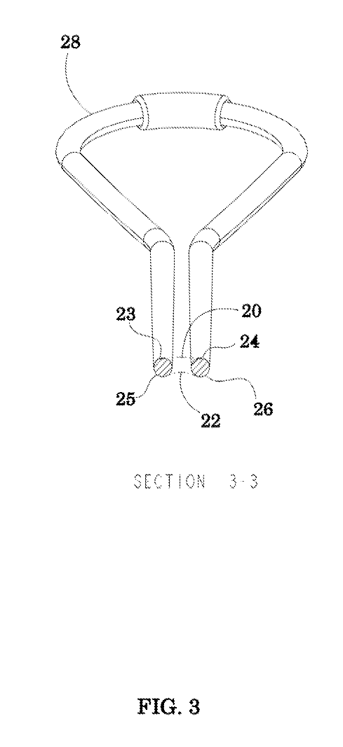

FIG. 3 illustrates a tapered flexible dual shaft 30 with a transverse cross-section 3 having a naturally occurring curvature element to adapt to the upper 20 and under 22 surface of the flexor tendon sheath and phalanx surfaces, Curvature 20 is defined by a first surface 23 and a second surface 24 that is adjacent to the first surface 23. Curvature 22 is defined by a first surface 25 and a second surface 26 that is adjacent to the first surface 25. This illustrates the benefits of a curvature element 20 and 22, in its longitudinal axis to match the convex outline of the volar aspect of the proximal and middle phalanx where the tightest pulleys make suture retrieval more challenging.

FIG. 1 illustrates an aperture 31 in the distal tip 29 large enough to easily accommodate any applicable suture size. The benefit is having the aperture 31 at the very tip, where it can easily be accessible but does not interfere with the function of the tip which works as a self locating feature.

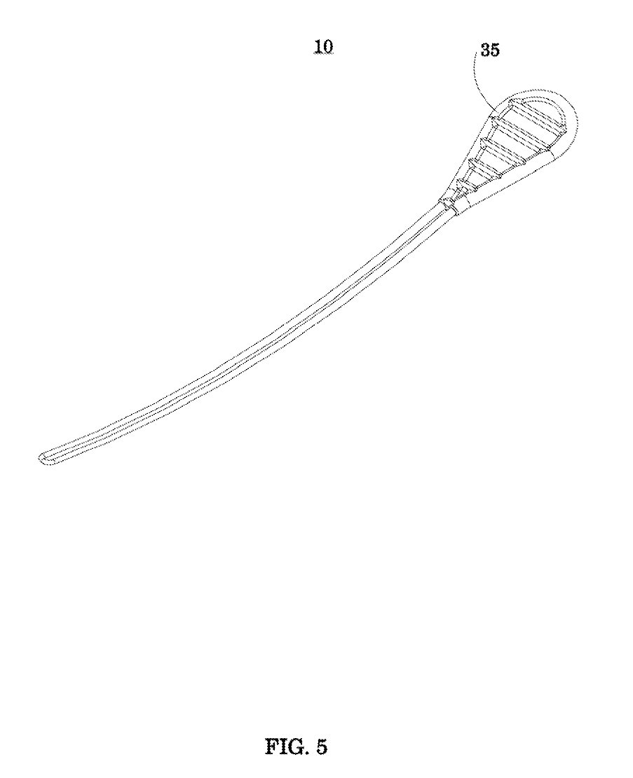

FIG. 5 illustrates an additional embodiment of the invention with a solid handle 35 e.g. over-molded with a suitable material.

The foregoing description is considered as illustrative only of the principles of the invention. Further, since numerous modifications and changes may readily occur to those skilled in the art, it is not desired to limit the invention to the exact construction and operation shown and described, and accordingly, all suitable modifications and equivalents are considered to fall within the scope of the invention. Various features and advantages of the invention are set forth in the following claims.

All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

1. A suture passer device, comprising:
a dual shaft retriever, the shafts being substantially U-shaped at the distal tip, the shafts having a tapered central region which extends out to a looped handle portion at the proximal end, a first shaft extending from and tapering from the central region and a second shaft extending from and tapering from the central region, opposite and parallel to the first shaft, the central region smallest of diameters and looped for receiving a suture, the central region lying at the end of a gentle arc, the handle portion lying opposite of the central region at opposite end of arc.

2. The device of claim 1, wherein the handle region is a loop with a filled inner portion making the handle a solid.

3. The device of claim 1, wherein the shaft consist of a flexible memory metal.

4. A method for retrieval of a severed flexor tendon in the hand comprising: a device having a tapered dual shaft, a distal end, a proximal end and a gently curved arced length between the distal and proximal ends of the shafts, the shaft portion lying between the handle portion and distal tip portion capable of passage through a flexor tendon sheath, the shaft having a convex upper surface to contact an upper surface of an interior surface, of the tendon sheath, the shaft having a convex under surface opposite the upper surface to contact an interior under surface of the tendon sheath and or an outer surface of the tendon, the shafts having a taper from the central portion of the shafts to the handle portion. The shaft material comprised of a flexible memory metal e.g. but not limited to Nitinol.
